(19) United States
(12) Patent Application Publication
(10) Pub. No.: US 2026/0216068 A1
(43) Pub. Date: Jul. 30, 2026

(54) URIC ACID LIPOSOMES

(71) Applicant: FreeOx Biotech, S.L., Barcelona (ES)

(72) Inventor: Pedro RAMOS CABRER, San Sebastián (ES)

(21) Appl. No.: 19/575,333

(22) Filed: Mar. 23, 2026

Related U.S. Application Data
(63) Continuation-in-part of application No. 18/693,714, filed on Oct. 25, 2022, now Pat. No. 12,599,605.

(30) Foreign Application Priority Data
Oct. 29, 2021 (EP) 21382979.9

(51) Int. Cl. A61K 9/127; A61K 9/1277; A61K 31/522

(52) U.S. Cl. CPC A61K 9/127; A61K 9/1277; A61K 31/522

(57) ABSTRACT

A liposome that encapsulate uric acid wherein the liposome has a lipid bilayer and wherein the uric acid is obtained without the use of lithium or potassium ions; methods for its preparation and uses thereof.

### CROSS-REFERENCE TO RELATED APPLICATIONS

This application is a continuation in part of U.S. patent application Ser. No. 18/693,714, filed Mar. 20, 2024, which is a 371 of PCT/EP2022/079717, filed Oct. 25, 2022, which claims the benefit of priority from European Patent Application No. 21382979.9, filed Oct. 29, 2021, the contents of each of which are incorporated herein by reference.

### TECHNICAL FIELD

The present invention relates to the pharmaceutical and biomedical field, more specifically to new liposomes that encapsulate uric acid and their manufacturing process. Additionally, the present invention also relates to the treatment of cerebrovascular diseases, more preferably stroke.

### BACKGROUND OF THE INVENTION

Cell death after stroke is the result of the complex interaction of excitotoxicity, acidosis, inflammation, oxidative stress, periinfarct depolarization, and apoptosis.

The term apoptosis is used synonymously with programmed cell death (hereinafter, PCD); however, apoptosis was originally defined as a set of morphological changes that occur after PCD. In developing neurons, these changes include chromatin condensation and cleavage and the formation of so-called apoptotic bodies. These changes are different from the morphological changes that characterize inflammation due to necrosis of the cytoplasmic organelles and the rupture of the mitochondrial and cytoplasmic membrane.

A mild ischemic injury usually induces cell death through an apoptotic-like mechanism rather than necrosis. Activators of apoptosis include oxygen free radicals, binding to death receptors, DNA damage, protease activation, and ion balance imbalance. Several experimental studies have shown that inhibition of apoptosis reduces the severity of ischemic injury.

Activation of caspases is a consequence of the activation of the intrinsic apoptosis pathway in which the mitochondria plays a fundamental role. Mitochondrial dysfunction and the opening of the mitochondrial transient permeability pore can result in caspase activation through the exit of Cytochrome C into the cytoplasm; however, there are other different mechanisms by which mitochondrial dysfunction can contribute to ischemic neuronal death. Severely damaged mitochondria may be unable to maintain the electrochemical gradient necessary for respiration and glucose oxidation. Thus, mitochondrial dysfunction can aggravate ischemic injury by exacerbating energy failure. Dysfunctional mitochondria also produce oxygen or nitrogen free radicals and non-radical substances that damage other cell organelles and DNA. Therefore, treatments that prevent mitochondrial dysfunction could be a more powerful neuroprotective strategy than caspase inhibition.

High Ca², Na⁺ and ADP (adenosine diphosphate) intracellular levels cause the mitochondria to produce harmful levels of reactive oxygen or nitrogen species. Unlike other organs, the brain is especially vulnerable to reactive oxygen or nitrogen species because neurons have relatively low levels of endogenous antioxidants. The abundance of oxygen or nitrogen radicals causes the destruction of cellular macromolecules and participates in signaling mechanisms that cause apoptotic cell death. Ischemia activates nitric oxide synthase (hereinafter NOS) and increases the generation of nitric oxide (hereinafter NO), which combines with superoxide to produce peroxynitrite, a potent pro-oxidant agent. NO production and oxidative and nitrosative stress are also linked to the overactivation of poly (ADP-ribose) polymerase-1 (hereinafter, PARP-1), a DNA repair enzyme.

After reperfusion, there is an increase in the production of superoxide, NO and peroxynitrite. The formation of these radicals in the vicinity of blood vessels plays an important role in the injury induced by reperfusion and in the appearance of insufficient reperfusion despite adequate proximal recanalization (non-reflux phenomenon). These radicals activate metalloproteases (hereinafter, MMP), which degrade collagen and laminins in the basal lamina, disrupt the integrity of the vascular wall, and increase the permeability of the blood-brain barrier (hereinafter, BBB). Oxidative and nitrosilative stress also activate the recruitment and migration of neutrophils and other leukocytes into the cerebral vasculature, which release enzymes that further increase degradation of the basal lamina and vascular permeability. These events can lead to parenchymal hemorrhage, vasogenic cerebral edema, and leukocyte infiltration within the brain. Oxidative and nitrosative stress constricts the pericytes or muscle cells that surround the capillaries and prevents adequate perfusion of microcirculation despite the normalization of blood circulation in leptomeningeal vessels.

Uric acid is a powerful antioxidant agent that blocks reaction between superoxide anion and nitric oxide, which damages cells by nitrosylating tyrosine residues from proteins. Plasma concentration of uric acid is almost 10 times higher than that of other antioxidant substances, such as vitamins C or E, and its antioxidant capacity is higher. In addition, uric acid prevents degradation of extracellular superoxide dismutase, an essential enzyme for normal endothelial function. In hippocampal cell culture, uric acid protects against excitotoxic glutamate damage, stabilizing calcium homeostasis and preserving mitochondrial function. Also, uric acid has shown inhibition of the Fenton reaction. Beyond its antioxidant effects, uric acid acts on transcription factors as a therapeutic target. Thus, uric acid activates the nuclear factor erythroid 2-related factor2/heme oxygenase 1 (Nrf2/HO-1) pathway and has a positive regulation in the expression of brain-derived neurotrophic factor (BDNF), and nerve growth factor (NGF).

In adult rat, administration of uric acid 24 hours before occlusion of the middle cerebral artery or 1 hour after reperfusion significantly reduces resulting cerebral infarction, suppresses accumulation of reactive oxygen species and decreases lipid peroxidation (Yu Z F, et al. Uric acid protects neurons against excitotoxic and metabolic insults in cell culture, and against focal ischemic brain injury in vivo.; J Neurosci Res 1998; 53: 613-25). Uric acid administration is neuroprotective in a rat thromboembolic model of focal cerebral ischemia and this neuroprotective effect is synergistic with respect to the beneficial effect achieved by rtPA (Romanos E, Planas A M, Amaro S, Chamorro A. Uric acid reduces brain damage and improves the benefits of rt-PA in a rat model of thromboembolic stroke. J Cereb Blood Flow Metab. 2007; 27:14-20).

There are studies that evidence relationship between higher uric acid levels in the blood at the time of a cerebral infarction and the lower neurological severity caused by it.

Additionally, the recent URICO-ICTUS study (phase 2b/3 clinical study) showed that the use of uric acid in combination with standard thrombolytic treatment (alteplase) is safe. In any case, combined therapy in this study did not show statistically significant effect, so conclusion of the study is that no significant change was observed in proportion of patients with excellent results at 90 days (Chamorro A, Amaro S, Castellanos M, Segura T, Arenillas J, Marti-Fábregas J, Gállego J, Krupinski J, Gomis M, Cánovas D, Carné X, Deulofeu R, Román LS, Oleaga L, Torres F, Planas A M; URICO-ICTUS Investigators. Safety and efficacy of uric acid in patients with acute stroke (URICO-ICTUS): a randomized, double-blind phase 2b/3 trial. Lancet Neurol. 2014; 13:453-60). On the other hand, PCT patent application WO2010112113A1 discloses the combined use of uric acid and citicoline for the treatment of stroke, demonstrating its effects in ischemic model cell cultures.

Additionally, the PCT patent application WO2018206826A1 demonstrates the usefulness and efficacy of uric acid for the treatment of cerebral infarction in patients treated by mechanical thrombectomy.

Despite the above, it should be noted that the use of uric acid as a therapeutic agent presents a series of problems that limit its use. On the one hand, uric acid has limited solubility with a tendency to crystallize, which complicates the logistics of its use as a pharmaceutical formulation.

On the other hand, the presence of high levels of free uric acid in blood (normally excreted via the kidneys) is usually the cause of the appearance of kidney stones and gout processes, due to the accumulation of urate crystals.

On the other hand, uric acid in the blood has a limited capacity to cross the blood-brain barrier and access the brain parenchyma.

An alternative to the use of free uric acid as a therapeutic agent is the use of therapeutic agents transport systems or platforms (nanomaterials) and controlled release (TLCs), which facilitate solubilization and stability in solution of uric acid (stability of pharmaceutical formulations, allowing adequate logistics for its use in the clinical setting) and its administration to the body in therapeutically relevant doses without exceeding pathological limits of free uric acid in blood. In the state of the art, such solutions for uric acid have not yet been described.

Therefore, given what has been explained above, in the state of the art there is still a need for systems or transport platforms and controlled release (TLCs) for uric acid, which facilitate: solubilization and stability in solution of said uric acid; its administration to the body in therapeutically relevant doses without exceeding pathological limits of free uric acid in the blood; and that allow or facilitate uric acid to cross the blood-brain barrier.

### SUMMARY OF THE INVENTION

The inventors of the present invention, after extensive and exhaustive experiments, have managed to generate liposomes that effectively encapsulate uric acid and that consequently allow solving the problems and needs present in the state of the art and described above:
1) Facilitate preparation of time-stable uric acid solutions under normal storage conditions.
2) Facilitate controlled and sustained release of therapeutic doses of uric acid into the bloodstream after intravenous administration.
3) Facilitate transfer of uric acid through the blood-brain barrier for its release in the brain parenchyma.
4) Improve the solubility of free uric acid. In this sense, the US publication 2024/0390381 discloses the dissolution of the starting uric acid material by a process using lithium and potassium ions, which involves a strong ionic solution, for the obtention of the liposomes containing the active agent. However, the present invention provides an alternative solution without the need for using lithium or potassium ions.
5) Improved uric acid stability.
6) Greater ease in synthesis or manufacture of uric acid.

Finally, as will be apparent from examples included herein liposomes with encapsulated uric acid have been shown to be more effective in treating stroke than free uric acid.

### BRIEF DESCRIPTION OF THE DRAWINGS

FIG. 1 shows the liposomes stability of the present invention over time when stored at room temperature.

FIG. 2 shows the experimental scheme of the experiment in Example 4, discussed further below.

FIG. 3 shows the volume of cerebral infarction (in percentage with respect to volume of the corresponding hemisphere (corrected for edema)) obtained in the experiment in Example 4.

FIG. 4 shows the result of the neurological tests (neuroscore) obtained in the experiment in Example 4.

FIGS. 5A, 5B, 5C and 5D show fluorescence confocal microscopy images obtained in Example 5 of mouse ischemic brains.

FIG. 6 shows a PXRD pattern conducted on the co-crystal of uric acid and L-lysine (Polymorph A).

### DETAILED DESCRIPTION OF THE INVENTION

The present invention describes an alternative process using co-crystals of uric acid as starting material, avoiding the use of such lithium and potassium ions used in US publication 2024/0390381, for a similar yield, with the technical effect of facilitating the solubilization of the uric acid in a surprising extent.

Therefore, in a first aspect, the present invention relates to liposomes that encapsulate uric acid.

In a second aspect, the present invention relates to a process for the manufacture of liposomes of the present invention.

In a third aspect, the present invention relates to liposomes that encapsulate uric acid obtained by the process for the manufacture of liposomes of the present invention.

In a further aspect, the present invention relates to a pharmaceutical composition comprising the present invention liposomes.

In a fifth aspect, the present invention provides a pharmaceutical composition or liposomes, both according to the present invention, for use as a medicine, more preferably for use in prevention, amelioration and/or treatment of a cerebrovascular disease, even more preferably for use in prevention, amelioration and/or treatment of stroke.

In a sixth aspect, the present invention relates to use of a pharmaceutical composition or liposomes, both according to the present invention, for the preparation of a medicament for prevention, improvement and/or treatment of a neurovascular disease, even more preferably for prevention, improvement and/or treatment of stroke.

In a final aspect, the present invention refers to a prevention method, improvement and/or treatment of a neurovascular disease (preferably, stroke) in a patient in need thereof, which comprises the administration of a pharmaceutical composition or liposomes, both according to the present invention, to said patient.

As used herein, “cerebral infarction”, “stroke” and “cerebrovascular accident” are used interchangeably, interchangeably and equivalently and refer to any pathology or clinical situation that implies that a part of the brain is left without blood irrigation.

As used herein, “patient” and its plural are used to refer to mammals, preferably humans, suffering from stroke, regardless of their sex and age and regardless of whether they have other pathologies (diagnosed or not).

As used herein, “liposome” and its plural acquire the meaning that they commonly have in the state of the art, that is, they are small artificial vesicles of spherical shape that can be created from cholesterol and non-toxic natural phospholipids. Liposomes have a simultaneous hydrophobic and hydrophilic character, and their hydrodynamic diameter can vary from 25 to 2500 nm (0.025 to 2.5 μm). Based on their size and bilayers number that form them, liposomes can also be classified into: (1) multilamellar vesicles (MLV) and (2) unilamellar vesicles. Unilamellar vesicles, in turn, can be classified into: (1) large unilamellar vesicles (LUV) and (2) small unilamellar vesicles (SUV).

As used herein, “hydrodynamic diameter” takes on the meaning that it commonly has in the state of the art. Whenever this variable is mentioned herein, it is measured using the DLS (Dynamic Light Scattering) technique, carried out in water at a temperature of 25° C.

Therefore, as indicated above, in a first aspect, the present invention relates to liposomes that encapsulate uric acid.

In the most preferred embodiment, the present invention relates to liposomes that encapsulate uric acid.

In a preferred embodiment, the liposomes of the present invention encapsulate between 6*10⁻²⁰ and 6*10⁻¹⁸ uric acid moles per liposome, more preferably 6.24*10⁻¹⁹ uric acid moles per liposome.

Additionally, liposomes of the present invention can be any type of liposome known in the state of the art. More preferably, liposomes of the present invention are unilamellar liposomes, even more preferably small unilamellar vesicles (SUV).

Also preferably, liposomes of the present invention have a hydrodynamic diameter of between 80 and 140 nm, more preferably 110 nm. Said hydrodynamic diameter is obtained by the DLS (Dynamic Light Dispersion) technique, carried out in water at a temperature of 25° C.

In a preferred embodiment, the liposomes of the present invention have a phase transition temperature higher than 45° C., more preferably a phase transition temperature between 45° C. and 70° C., even more preferably a phase transition temperature higher than 45° C. and lower than 70° C., even more preferably a phase transition temperature of 55° C.

Also preferably, liposomes of the present invention have a positive surface charge, more preferably zeta potential of liposomes of the present invention is greater than 0 mV and less than 15 mV, even more preferably 10 mV. Said zeta potential is obtained using a DLS (Dynamic Light Dispersion) equipment, measuring in water at a temperature of 25° C.

Liposomes of the present invention have a lipid bilayer with a suitable composition to allow liposomes synthesis and stability, for encapsulation of uric acid and for its release (preferably, at the site of interest, more preferably in the brain and, preferably in a sustained way over time). In a preferred embodiment, liposomes of the present invention comprise positively charged double chain phospholipids and cholesterol. Therefore, preferably, liposomes of the present invention are unilamellar and present a lipid bilayer that encapsulates uric acid, said lipid bilayer comprising double-chain phospholipids and at least one positively charged cholesterol derivative (more preferably, said lipid bilayer consisting of double chain phospholipids and at least one positively charged cholesterol derivative).

Preferably, the at least one positively charged cholesterol derivative is a positively charged cholesterol derivative. More preferably, the positively charged cholesterol derivative is dimethylaminoethane-carbamoyl-cholesterol hydrochloride (Dimethylaminoethane-Carbamoyl-cholesterol (3β-[N-(N′,N′-dimethylaminoethane)-carbamoyl]cholesterol hydrochloride, hereinafter DC-cholesterol).

Preferably, double-chain phospholipids comprise a combination of phospholipids having one or more chains of a long-chain polymer that hinder liposomes opsonization (opsonin binding to liposome) in blood (more preferably, a linked chain to its polar head) and phospholipids that do not have polyethylene glycol attached to its polar head. In a preferred embodiment, in the liposomes of the present invention:
between 2.5% and 10% molar of the double-chain phospholipids are phospholipids with one or more chains of a long-chain polymer that hinder liposomes opsonization in blood attached to their polar head; more preferably, 7.5% molar of double-chain phospholipids are phospholipids with long-chain polymer chains that hinder liposomes opsonization in blood; and
the rest of the double-chain phospholipids are phospholipids without polyethylene glycol attached to their polar head, that is, between 90 and 97.5% molar of the double chain phospholipids are phospholipids without polyethylene glycol attached to their polar head, more preferably 92.5% mol are phospholipids without polyethylene glycol attached to its polar head.

Long chain polymer that hinders liposomes opsonization in blood is preferably selected from: polyethylene glycol (PEG), Polyoxazolines (POX), Polyvinylpyrrolidinones (PVP), Polyglycerols (PG), Polyacrylamides (PAA, NIPAM, PHPMA, PNIPAM), Polysaccharides, Polyaminoacids or combinations thereof, more preferably the Long-chain polymer that hinders liposomes opsonization in blood is polyethylene glycol.

Preferably, in double-chain phospholipids with one or more polyethylene glycol chains attached to their polar head (more preferably, a polyethylene glycol chain attached to its polar head), each of polyethylene glycol chains has a molecular weight of between 1000 and 5000 Da, more preferably a molecular weight of 2000 Da.

Also preferably, double chain phospholipids with one or more polyethylene glycol chains attached to their polar head (more preferably, a polyethylene glycol chain attached to their polar head) are selected from 16: 0 PEG2000 PE (or 1,2 dipalmitoyl-sn-glycero-3-phosphoethanolamine-N-[methoxy (polyethylene glycol)-2000]), 18: 0 PEG2000 PE (or 1,2-distearoyl-sn-glycero-3-phosphoethanolamine-N-[methoxy (polyethylene glycol)-2000]), 18: 0 PEG5000 PE (or 1,2-distearoyl-sn-glycero-3-phosphoethanolamine-N-[methoxy (polyethylene glycol)-5000]) or combinations thereof. In the most preferred embodiment, double-chain phospholipids with one or more polyethylene glycol chains attached to their polar head (more preferably, a polyethylene glycol chain attached to its polar head) are selected from 16: 0 PEG2000 PE, 18: 0 PEG2000 PE, or combinations thereof, even more preferably, double chain phospholipids with one or more polyethylene glycol chains attached to their polar head (more preferably, a polyethylene glycol chain attached to their polar head) are 18: 0 PEG200 PE.

Double-chain phospholipids without polyethylene glycol attached to its polar head are preferably phospholipids derived from phosphatidylcholine, neutral (zwitterionic) and each of its two chains having 16 to 18 carbon units. More preferably, double chain phospholipids without polyethylene glycol attached to its polar head are selected from DSPC (18: 0 PC or 1,2-distearoyl-sn-glycero-3-phosphocholine), DPPC (16: 0 PC or 1,2-dipalmitoyl-sn-glycero-3-phosphocholine)), 17: 0 PC (or 1,2-diheptadecanoyl-sn-glycero-3-phosphocholine) or combinations thereof. In the most preferred embodiment, double chain phospholipids without polyethylene glycol attached to its polar head are DSPC.

Preferably, in liposomes of the present invention, molar fraction ratio between double chain phospholipids and positively charged cholesterol (preferably DC-cholesterol) is between 0.6: 0.4 and 0.75: 0.25, more preferably, molar fraction ratio of double chain phospholipids to positively charged cholesterol (preferably DC-cholesterol) is 0.667: 0.333.

Therefore, in the most preferred embodiment, in liposomes of the present invention, their membrane (lipid bilayer) consists of DSPC: DC-Cholesterol: 18: 0 PEG2000-PE with a ratio, in molar fractions, of 0.617: 0.333: 0.050.

Preferably, liposomes of the present invention are stable at room temperature for at least 5 days, more preferably, at least 7 days, more preferably, at least 12 days, more preferably at least 15 days, even more preferably at least 21 days. In this period of time, liposomes conserve at least 80% of encapsulated uric acid in relation to encapsulated uric acid that initially presented.

As supported by the results of the examples included herein, liposomes of the present invention make it possible to solve the problems present in the state of the art and, consequently:
1) They facilitate preparation of time-stable uric acid solutions under normal storage conditions (at least 21 days at room temperature without signs of precipitation and keeping the transparence of the preparation).
2) They facilitate handling and conservation of uric acid.
3) They facilitate or allow controlled and sustained release of therapeutic doses of uric acid into the bloodstream after intravenous administration.
4) They facilitate transfer of uric acid through the blood-brain barrier for its release in the brain parenchyma.
5) They allow to overcome solubility of free uric acid.
6) They allow to improve stability of uric acid.
7) They provide ease in the synthesis or manufacture of uric acid.
8) They provide a superior therapeutic effect compared to the use of free uric acid.

In a second aspect, as indicated above, the present invention relates to a process for preparation or manufacture of liposomes that encapsulate uric acid comprising the steps of:
a) Form a lipid film;
b) Rehydrate the lipid film formed in step a) to obtain liposomes;
c) Extrude obtained liposomes in step b); and
d) Filter obtained liposomes in step c),
   characterized in that:
Steps a) to d) are carried out free of calcium ions;
Purified water is used as suitable solvent.
To rehydrate the lipid film, in a second phase, a solution of a uric acid-lysine co-crystal is used. Once the crystals are solved, the process is maintained at the phase transition temperature (Tₘ) of the lipids or above. The solvent is then added to the lipid film until the film is completely dissolved.
In a third phase an extrusion system is heated to Tₘ. With the extruder balanced at a that temperature, The extruder is filled with the aqueous lipid mixture, allowing the temperature to equilibrate, for the extrusion of the mixture through a membrane, repeating the process several times.
In a fourth phase the extruded liposomes are filtrated through a centrifugal filter.

In step a) of the process of the present invention, a lipid bilayer is formed using desired or suitable lipid components, that is, it is formed using the desired components for the lipid bilayer of the liposomes. In this sense, in relation to lipid bilayer and its composition, everything indicated above in the first aspect of the present invention applies.

In extrusion step c), liposomes are extruded so that desired size liposomes are obtained, more preferably, liposomes are passed (extruded) through extrusion membranes with the appropriate pore size to obtain liposomes of the size wanted. Preferably, extrusion is carried out serially from larger pore size membranes to smaller pore size membranes until the desired liposome size is reached. For example, and preferably, twice through 0.4-micron pore size membranes (preferably polycarbonate), then 4 times using a 0.2-micron pore size membrane (preferably polycarbonate), and finally 8 times using a 0.1-micron pore size membrane (preferably polycarbonate) to obtain nominal size liposomes (hydrodynamic diameter) around 100 nanometers (0.1 microns).

In filtration step d), components not incorporated in liposomes are removed and, if deemed appropriate, the medium can be changed. Step d) can be carried out by any known method in the state of the art, more preferably it is carried out by molecular mass cutting filtration (filtration through membrane by centrifugation) or by dialysis.

In a preferred embodiment, HPLC (high performance liquid chromatography) grade water is used throughout the method.

Also, in a more preferred embodiment of the process of the present invention, the entire process is carried out free of divalent ions.

Additionally, preferably, steps a) to d) of the process of the present invention are carried out sodium and/or carbonate ions free. Most preferably, the entire method is carried out sodium and carbonate ions free.

In a preferred embodiment of the process of the present invention, after step d), process comprises a step of medium substitution in which liposomes are dissolved.

In another preferred embodiment of the process of the present invention, after step d), process comprises a lyophilization step. Also preferably, after lyophilization step, the process of the present invention comprises a reconstitution step.

In a more preferred embodiment, in the process of the present invention all steps are carried out calcium ions free.

In everything not detailed above, the process of the present invention is as liposomes manufacture or preparation processes of the state of the art.

Therefore, preferably the liposomes manufacture or preparation process that encapsulate uric acid of the present invention comprises:

### Phase 1: Lipid Film Formation:

1) Allow reagents to reach room temperature.
2) Clean the material to be used or use sterile material.
3) Dry glassware to be used (preferably under a nitrogen or argon gas stream).
4) Weigh the required amount of double-chain phospholipids and positively charged cholesterol (according to explained above in the first aspect of the present invention) and dissolve them in an organic solvent (preferably, in a CH₃Cl: MeOH (chloroform-methanol, 6: 1)) mixture.
5) Place obtained solution in step 4 on a rotary evaporator and completely evaporate the organic solvent (preferred working conditions are: bath at 30° C., rotation speed of about 140 rpm (revolutions per minute) and controlled pressure drops up to 200 mbar (20 kPa), and from this point on, reduce the pressure very slowly (or increase the vacuum very slowly) to the minimum possible over a period of 10 minutes).

Is very important that once the solvent evaporates, a homogeneous lipid film remains at the bottom of the used container (preferably frosted pear-shaped flask). If not, it will be necessary to repeat steps 4 and 5 above.
6) Hold the container with step 5 film (preferably frosted pear-shaped flask) at maximum vacuum for at least 15 minutes.
7) Break the vacuum slowly and place the container for a minimum of 1 hour under stream of gas (preferably nitrogen or argon) or keep it in a high vacuum desiccator for between 12 and 24 hours, to favor total evaporation of organic solvents.

### Phase 2: Lipid Film Rehydration

8) A solution of uric acid co-crystal formed with a co-former molecule, preferably an amino acid, more preferably lysine, in a suitable solvent, preferably double distilled and deionized water or more preferably HPLC purified water. The solution is preferably prepared at room temperature. Then adjust the pH to a 7.0-7.4 range typically using a a strong base or strong acid.
9) The solution of the uric acid to be encapsulated is then heated to the phase transition temperature (Tₘ) of the lipids or above, maintaining this temperature throughout the process.
10) Then, the solution is added to the flask containing the lipid phase until the film completely dissolved.

### Phase 3: Liposome Extrusion

11) With extruder balanced at working temperature, wash it 3 times with excess of solvent and extrude the solution obtained in step 10 using one or more membranes with appropriate pore size depending on the wanted liposomes size.
   Phase 4: Liposome filtration
12) Filter the extruded solution of step 11) through filtration systems with cut by molecular mass or through dialysis processes.

As stated above, the entire method is carried out calcium ions free (and, preferably, the entire method is also carried out sodium and carbonate ions free).

All preferred embodiments explained above for the process of the present invention apply in this case.

Preferably, step 11 indicated above comprises:
a) Assemble a 0.4 microns pore size membrane (preferably polycarbonate) in the extruder and wet it with the solvent used in the lipid film rehydration phase 2.
b) Fill the extruder with the obtained solution of step 10 and allow the temperature equilibrating at a temperature above the phase transition temperature of the liposomes lipids, preferably at 65° C. and approximately 10 min.
c) Using gas stream (preferably nitrogen or argon) to force extrusion of the solution through the membrane. The sample is collected in a hot container, at working temperature (preferably, working temperature should never be lost).
d) Repeat extrusion process on the 0.4-micron membrane (steps 12 to 14).
e) Repeat the process preferably 4 times, now using a 0.2-micron pore size membrane, preferably polycarbonate.
f) Repeat extrusion process preferably for 8 times using a 0.1-micron pore size membrane, preferably polycarbonate, to obtain nominal size liposomes (hydrodynamic diameter) around 100 nanometers (0.1 microns).

It renders evident that a person skilled in the art will be able to adjust membrane sizes and repeats during extrusion in order to obtain the desired size liposomes.

In phase 4, individual components that have not been integrated into liposomes are eliminated and their final concentration is adjusted (adjustment of the final volume of solution). In a preferred embodiment, therefore, step 12 of the process of the present invention comprises:
a) Fill with the solvent a tube (preferably a centrifugal filtration unit) with a cut-off membrane of 30 kDa and filter it in a centrifuge (preferably 2 hours at 6000 g at room temperature). Preferably, this filtering should be carried out 3 times.
b) Pass the extruded liposome solution obtained in step 11 to centrifugal filtration unit previously washed, and centrifuge (preferably, 2 hours at 6000 g).
c) Adjust the final volume of liposome solution to the desired value (for example, 10 mL) and adjust the pH to the desired value (preferably 7.2-7.4), preferably using HCl (1 N) or NaOH (1 N).

As can be seen in examples included herein, process of the present invention allows the correct liposomes manufacture by encapsulating uric acid in 100% of cases, preventing precipitates from appearing and providing stable liposomes over time at room temperature (at less for 21 days and conserving 80% of encapsulated uric acid). Additionally, and surprisingly, liposomes obtained by the method of the present invention have a greater therapeutic effect than liposomes obtained by the method of the state of the art.

In a third aspect, the present invention relates to liposomes that encapsulate uric acid obtained by the method of the present invention (explained in the second aspect of the present invention).

Preferred embodiments and explanations given in the first and second aspects of the present invention are applicable to this third aspect of the present invention (with the necessary adaptations).

In a fourth aspect, as indicated above, the present invention refers to a pharmaceutical composition comprising liposomes according to first aspect of the present invention and/or obtained liposomes according to the process of the present invention (method explained in the second aspect of the present invention).

Preferably, the pharmaceutical composition of the present invention comprises a therapeutically effective uric acid concentration, more preferably between 0.5 and 10 mg/mL, more preferably 1.6 mg/mL.

Also preferably, the pharmaceutical composition of the present invention comprises a lipid concentration of between 10 and 2 mM, more preferably between 7 and 4 mM, more preferably between 4.3 and 4.7 mM, even more preferably 4.5 mM.

It is contemplated that the pharmaceutical composition of the present invention may be in any form known in the state of the art, provided that said form is compatible with the chosen administration form. Preferably pharmaceutical composition of the present invention is in liquid or lyophilized form, more preferably liquid. In cases wherein pharmaceutical composition of the present invention is in lyophilized form, is contemplated to be reconstituted with a suitable solution or solvent, preferably saline before use.

In a fifth aspect, the present invention provides a pharmaceutical composition or liposomes, both in accordance with the present invention, for use as medicine.

The pharmaceutical composition of the present invention is in accordance with what was explained above in the fourth aspect of the present invention.

The liposomes of the present invention are as explained above in the first or third aspect of the present invention.

More preferably, this fifth aspect of the present invention discloses a pharmaceutical composition or liposomes, both according to the present invention, for use in prevention, amelioration and/or treatment of a cerebrovascular disease, even more preferably, for its use in prevention, improvement and/or treatment of stroke.

Is contemplated that said stroke may be ischemic or hemorrhagic, more preferably the stroke is an ischemic stroke, even more preferably an ischemic stroke treated with thrombolytic drugs (e.g., alteplase and/or tenecteplase), an ischemic stroke treated by mechanical thrombectomy, or an ischemic stroke treated with thrombolytic drugs and mechanical thrombectomy. Is contemplated that these treatments (thrombolytic drugs, mechanical thrombectomy and any other stroke treatment that may be considered) may be prior, concurrent or subsequent to the use or administration of the pharmaceutical composition or liposomes of the present invention.

Therefore, the present invention contemplates that the pharmaceutical composition of the present invention or the liposomes of the present invention are used alone or in combination with other compounds (preferably active ingredients). In a preferred embodiment, they are used in combination with a thrombolytic agent, more preferably with a tissue plasminogen activator (hereinafter tPA) (for example, alteplase).

It is also contemplated that the pharmaceutical composition of the present invention or the liposomes of the present invention are used in combination with, for example, citicoline.

In relation to the foregoing, it is contemplated that the combined use is within the same composition or that is in the form of at least one additional composition. In the latter case, as indicated above, is contemplated that the pharmaceutical composition or liposomes of the present invention are administered before, at the same time or after the at least one additional composition.

In a preferred embodiment, the pharmaceutical composition or liposomes of the present invention are used in combination with a composition comprising tPA (preferably alteplase) and are used at the same time, that is, they are administered together, even more preferably, first the composition comprising tPA is administered and before the end of the administration thereof, the administration of the pharmaceutical composition or the liposomes of the present invention is started.

The dose of the pharmaceutical composition or liposomes of the present invention is a therapeutically effective dose.

In a preferred embodiment, a uric acid dose is between 10 and 20 mg/kg patient, 16 mg/kg of patient.

In a more preferred embodiment of this fifth aspect of the present invention, an administered dose is between 500 and 2000 mg uric acid, more preferably an administered dose is between 500 and 1000 mg uric acid, even more preferably an administered dose is 1000 mg uric acid.

The pharmaceutical composition of the present invention and the liposomes of the present invention, in this fifth aspect of the present invention, can be administered by any of the routes known in the state of the art. In a preferred embodiment, the pharmaceutical composition of the present invention and the liposomes of the present invention are administered intravenously.

The treatment is administered to a patient in need thereof, and said patient is in accordance with the above.

In a sixth aspect, the present invention relates to use of a pharmaceutical composition or liposomes, both according to the present invention, for the preparation of a medicament for prevention, improvement and/or treatment of a cerebrovascular disease.

In a preferred embodiment, cerebrovascular disease is stroke.

Embodiments explained in the fifth aspect of the present invention apply directly (with necessary adaptations) to this sixth aspect of the present invention.

In a final aspect, the present invention refers to a prevention method, improvement and/or treatment of a cerebrovascular disease in a patient in need thereof, which comprises the administration of a pharmaceutical composition or liposomes, both according to the present invention, to said patient.

In a preferred embodiment, cerebrovascular disease is stroke.

Embodiments explained for the fifth aspect of the present invention apply directly (with necessary adaptations) to this final aspect of the present invention.

To enable a better understanding, the present invention is described below in more detail with reference to the accompanying figures, which are filed by way of example, and with reference to the illustrative and non-limiting examples included below.

FIG. 1 shows the liposomes stability of the present invention over time when stored at room temperature, as indicated in Example 2 included below. FIG. 1 shows the uric acid percentage that remains encapsulated in the liposomes of the present invention at different time intervals, as a measure of stability of said liposomes. In this sense, a 100% value is established for encapsulated uric acid in liposomes on day 1 and the result for the rest of days is indicated based on or compared to the value on day 1. On y-axis, encapsulated uric acid in liposomes percentage is indicated (considering as 100% the amount of encapsulated uric acid in the liposomes on day 1). x-axis shows the time (storage) in days since the start of the experiment.

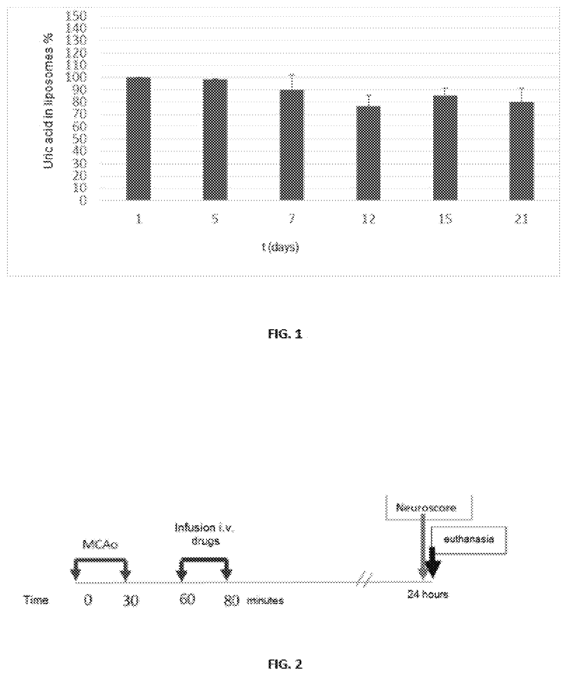

FIG. 2 shows the experimental scheme of the first experiment included in Example 4 below.

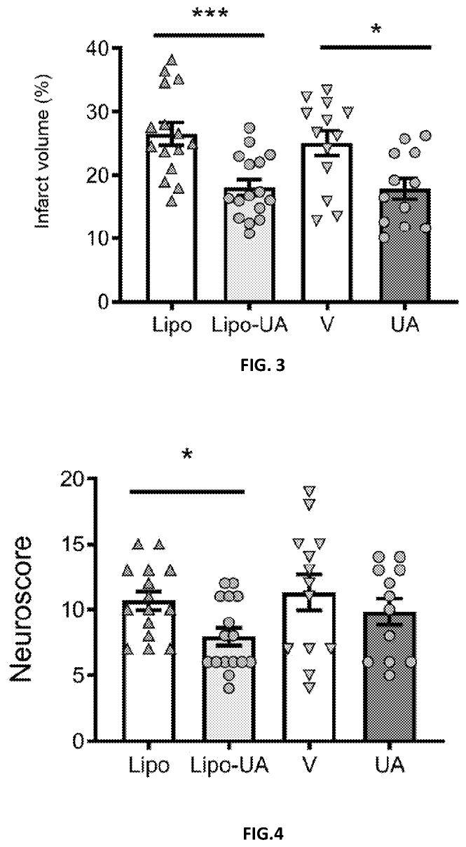

FIG. 3 shows the volume of cerebral infarction (in percentage with respect to volume of the corresponding hemisphere (corrected for edema)) obtained in the first experiment mentioned in example 4. y-axis reflects the volume of cerebral infarction (in percentage with respect to volume of the corresponding hemisphere (corrected for edema)) and x-axis shows the different experimental groups, from left to right, as indicated in example 4: Lipo (empty liposomes diluted in saline), Lipo UA (Liposomes according to the present invention, i.e., that encapsulate uric acid), V (vehicle) and UA (free uric acid solution).

FIG. 4 shows the result of the neurological tests (neuroscore) obtained in the first experiment mentioned in example 4. y-axis reflects the neuroscore and x-axis shows the different experimental groups, from left to right, as indicated in example 4: Lipo (empty liposomes diluted in saline), Lipo UA (Liposomes according to the present invention, i.e., that encapsulate uric acid), V (vehicle) and UA (free uric acid solution).

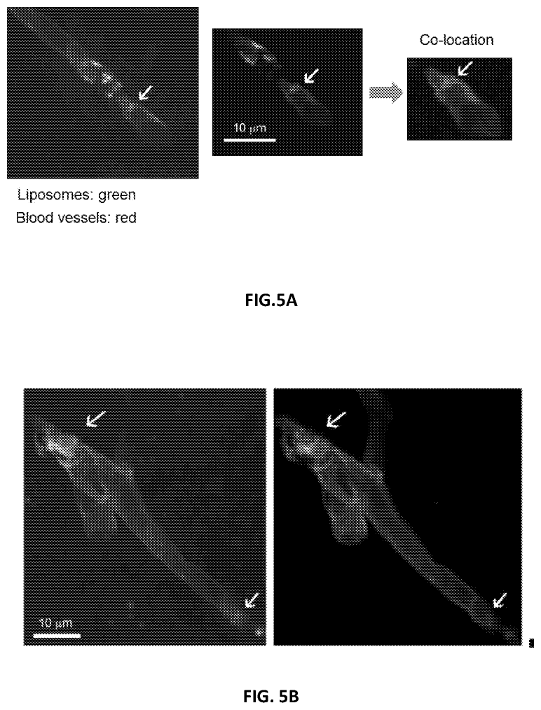

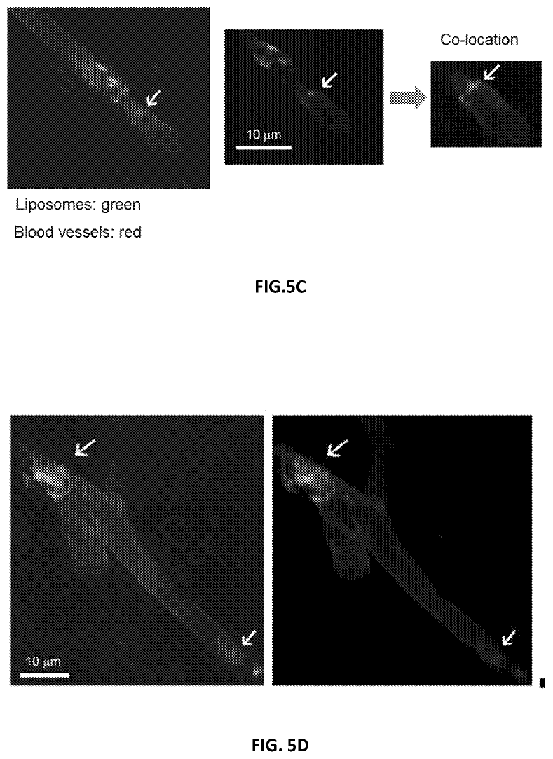

FIG. 5 shows fluorescence confocal microscopy images obtained in Example 5 of mouse ischemic brains. In FIGS. 5A and 5B images, cell nuclei are shown in blue, cerebral blood vessels in red and liposomes of the present invention in green. Is observed that the liposomes of the present invention co-localize or localize in the cerebral vessels of mice. In both FIG. 5A and FIG. 5B, the dates show the co-localization areas of liposomes and cerebral blood vessels. The corresponding images on the right in both FIGS. 5A and 5B show in gray the cerebral liposomes and blood vessels co-localization zone as calculated by image analysis with the ImageJ Fiji program. FIG. 5C is the same as FIG. 5A but in black and white. FIG. 5D is the same as FIG. 5B but in black and white.

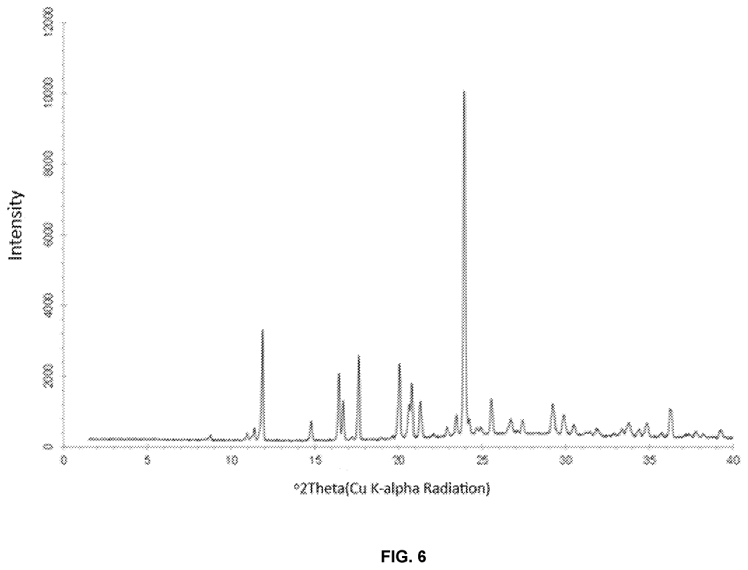

FIG. 6 shows a PXRD pattern conducted on the co-crystal of uric acid and L-lysine (Polymorph A).

### EXAMPLES

### Example 1. Preparation Liposomes Method by Encapsulating Uric Acid, Comparison of the State of the Art Method for the Preparation of Liposomes with the Method of the Present Invention

a) State of the art method for liposomes preparation (see, for example, Mulder W J, Strijkers G J, van Tilborg G A et al. Lipid-based nanoparticles for contrast-enhanced MRI and molecular imaging. NMR Biomed. 2006; 19 (1): 142-64; Needle J, Brea D, Argibay B, et al. Quick adjustment of imaging tracer payload, for in vivo applications of theranostic nanostructures in the brain. Nanomedicine. 2014; 10(4):851-8). “stealth or silent” or “long circulating blood time” liposomes preparation based on DSPC and cholesterol

For the preparation of DSPC liposomes (18: 0 PC, or 1,2-distearoyl-sn-glycero-3-phosphocholine), PEG2000-PE (1,2-distearoyl-sn-glycero-3-phosphoethanolamine-N-[methoxy (polyethylene glycol)-2000) and cholesterol are used in the amounts indicated in table 1, aiming at the preparation of liposomes containing a lipids total amount of 25 μmol (normally between 25-100 μmol are prepared).

TABLE 1
Molecular weight, molar fraction and total amount of each of
the liposome components to obtain 25 μmol of liposomes
in the liposome manufacturing process of the state of the art.

| | Molecular weight | | |
|---|---|---|---|
| components | (g/mol) | Amount (mg) | Molar fraction |
| DSPC (18:0 PC) | 790.1 | 12.18 | 0.617 |
| 18:0 PEG2000 PE | 2805.5 | 3.51 | 0.050 |
| Cholesterol | 386.6 | 3.22 | 0.333 |

All these components are commercially available (for example, from Avanti Polar Lipids with references 850365P, 880120P and 700001P).

The method carried out, briefly, was:

### Phase 1: Lipid Film Formation

1) Remove the reagents from the freezer (stored at −20° C.) and allow them to reach room temperature before opening the containers that contain them.
2) Clean a reaction flask (preferably pear-shaped) with milliQ water or similar (3×), followed by ethanol (3×) and acetone (3×). Clean in the same way a rotary evaporator and any other non-disposable glassware that needs to be used.
3) Dry the glassware to be used under nitrogen or argon gas stream.
4) Weigh the lipids and cholesterol required amount (see table 1), transfer them to the reaction flask and add 7 mL of CH₃Cl: MeOH (chloroform-methanol, 6: 1) mixture. Mix until the components are completely dissolved.
5) Place the reaction flask on a rotary evaporator and completely evaporate the organic solvent (typical working conditions are: bath at 30° C., rotation speed of about 140 rpm (revolutions per minute) and controlled pressure drops up to 200 mbar (20 kPa), and from this point on, reduce the pressure very slowly (or increase the vacuum very slowly) to the minimum possible over a period of 10 minutes). Is very important that once the solvent evaporates, a homogeneous lipid film remains at the bottom of the flask. If not, the reagents must be redissolved in 7 mL of the chloroform-methanol mixture and evaporated again.
6) Keep the flask with the film at maximum vacuum for at least 15 minutes.
7) Break the vacuum slowly and place the flask for a minimum of 1 hour under nitrogen or argon gas stream, or keep it in a high vacuum desiccator until the next day, to favor the total evaporation of organic solvents
   Phase 2: lipid film rehydration
8) Heat the solution with the active principle to be encapsulated (uric acid) to 65° C., maintaining this temperature throughout the process.
9) Place the reaction flask with the lipid film obtained in Phase 1 of the process in water bath until reaching working temperature and then add 7 mL of the solution prepared in step 8, and stir until the film is completely dissolved (the process can be facilitated adding a few units of glass beads of about 3 mm in diameter). Maintain stirring for about 10 min, with the flask always in the bath to avoid the temperature to drop below Tm.
   Phase 3: Liposome extrusion
10) Heat an extrusion system to the same temperature. In the present case, a 10 mL LIPEX Thermobarrel extruder from Evonik Industries was used, connected to a thermostatic water recirculation bath and a nitrogen gas stream, as the driving gas.
11) With the extruder balanced at a temperature between 60° C. and 80° C., wash it 3 times with an excess of solvent (prepared in step 8 explained above).
12) Mount a 0.4-micron pore size polycarbonate membrane on the extruder (Millipore) and wet it with the solvent.
13) Fill the extruder with the aqueous lipid mixture and allow the temperature to equilibrate (approximately 10 min).
14) Use a nitrogen or argon gas stream to force extrusion of the mixture through the membrane. The sample must be collected in a hot container, at a temperature between 60° C. and 80° C. (working temperature must never be lost).
15) Repeat the extrusion process on the 0.4-micron membrane once more.
16) Repeat the process (4 times) using now a membrane, in this case 0.2 microns and, finally, repeat the extrusion process (8 times) using a 0.1-micron pore size membrane (in total extrusion process involves 2×0.4 microns+4×0.2 microns and 8×0.1 microns), to obtain nominal size liposomes (hydrodynamic diameter) around 100 nanometers (0.1 microns).

### Phase 4: Liposome Filtration

The last step in liposome preparation consists of filtering the extruded solution through molecular mass cut filter systems (Amicon systems, for example) or subjecting them to dialysis processes. In this way, individual components that have not been integrated into liposomes are eliminated and their final concentration is adjusted (adjustment of the final volume of solution) and it is possible, if desired, to change the solvent used in the preparation of liposomes by another solvent of a hydrophilic nature (for example, by Serum or saline solution)
17) Fill a centrifugal filter unit (Amicon tube (or similar)) with 30 kDa cut-off membrane with solvent and filter it in a centrifuge (2 hours at 6000 g and at room temperature). Perform this filtering 3 times.
18) Pass the extruded liposome solution through the previously washed centrifugal filter unit (step 17) and centrifuge (2 hours at 6000 g).
19) Adjust the final volume of the liposome solution to the desired value (for example, 10 mL) and adjust the pH to the desired value (typically 7.2-7.4) with a few drops of HCl (1 N) or NaOH (1 N). The liposomes thus prepared, according to the state of the art, can normally be stored at room temperature (or at 4° C. if preferred or if encapsulating agent so requires) for a prolonged period of time from days to months, depending on the composition thereof.

In the specific case of uric acid liposomes, this state-of-the-art methodology was not adequate for two main reasons:
1) Low solubility of uric acid in organic solvents useful for the preparation of lipid films in the formation of liposomes (which prevented its incorporation into liposomes as part of the film, i.e., in step 4 described above) and the great instability which presents this compound in aqueous solutions (tendency to precipitate rapidly) under normal working conditions, made its incorporation into liposomes in stages 8-10 inefficient.
2) The fact that, after preparation, the liposomes encapsulating uric acid were not very stable in solution: In a period ranging from minutes to a few days, the precipitation of encapsulated uric acid was observed.

### b) Method of the Present Invention

The process of the present invention as indicated above is able to obtain uric acid liposomes with two main features:
1) That they are stable in solution at 4° C. or higher (room temperature) temperatures for a period of at least 21 days.
2) That said liposomes present a minimum amount of uric acid of 1.6 mg/ml (or 9.5 mM) in solution.

The manufacturing process of the liposomes that encapsulate uric acid of the present invention presented the following modifications with respect to the art:

1) For the lipid film formation, the steps explained above in the state of the art method were followed, but with the following composition for the lipid bilayer or liposome membrane: DSPC: DC-Cholesterol: 18:0 PEG2000-PE (0.617: 0.333: 0.050).

TABLE 2
Molecular weight, molar fraction and total amount of
each of the liposome components to obtain 100 μmol
of liposomes in the method of the present invention.

| | Molecular weight | | |
|---|---|---|---|
| components | (g/mol) | Amount (mg) | Molar fraction |
| DSPC (18:0 PC) | 790.1 | 48.72 | 0.617 |
| 18:0 PEG2000 PE | 2805.5 | 14.04 | 0.050 |
| DC-Cholesterol | 537.26 | 17.89 | 0.333 |

### 2) Preparation of the Uric Acid Solution:

To rehydrate the lipid film (Stage 2, steps 8-10 of the state of the art process) it was necessary to use an uric acid solution from the co-crystals of uric acid and L-lysine having a PXRD comprising a 2⊖ angle at value of 14.80 and six relevant 2⊖ angle values at 11.88, 16.44, 16.68, 17.61, 20.04, 20.63, 20.80, 21.30, 23.93, 25.56, 29.21, 34.81, and 36.25, with a ±0.1 tolerance in the 2⊖ angle values, preferably a ±0.2 tolerance (FIG. 6), corresponding to polymorph A as described in application PCT/EP2025/0830546.

A clear solution of uric acid was obtained from dissolving the uric acid co-crystals with the purified water, with no turbidity; the pH was lowered very slowly to 7.2-7.4 using a few drops of HCl 1M (solution without calcium traces).

The solution was stable at room temperature (and at 4° C.) for days. With this solution, the lipid film formed in phase 1 was rehydrated, following steps 8-10 as previously described (Working temperature T=65° C.) (i.e., as in the method for manufacturing or preparation liposome of the state of the art).

### C) Liposomes Extrusion and Filtration (Phases 3 and 4)

Extrusion followed the same steps as those indicated in the state of the art method, and purified water was used as solvent.

Filtration followed the same steps as those indicated in the state of the art method, using purified water as solvent.

Following all these modified steps, a clear solution (with no turbidity) of uric acid liposomes (which encapsulate said uric acid) was obtained, which had about 11 mM concentration of said uric acid (the exact amount will depend on the performance of the synthesis process).

### Results:

26 batches of uric acid liposomes were prepared.
1—liposomes preparation according to the conventional method of the state of the art explained above in this example, including the use of phosphate buffer saline as a vehicle
   Batches: 3 Batches with Problems: 3 Failed Batches: 100%
2—liposomes preparation according to the conventional method of the state of the art explained above in this example, but with pH control by dissolving uric acid and the use of Saline Serum as a vehicle
   Batches: 10 Batches with Problems: 4 Failed Batches: 40%
3—Preparation of liposomes according to the conventional method of the state of the art explained above in this example, with pH control when dissolving uric acid and of the final solution of liposomes, and the use of HPLC water in the synthesis (material washes made with MiliQ water)
   Batches: 7 Batches with Problems: 1 Failed Batches: 14%
4—Process of the present invention (the entire process done with HPLC water, including material washing)
   Batches: 6 Batches with Problems: 0 Failed Batches: 0%

Observed problems in the cases of batches 1 to 3 were basically formation of uric acid crystals in suspension and precipitation, at different times, after preparation. Surprisingly, with the process of the present invention, all these problems were solved, and all the manufactured batches were successful.

### Example 2: Process of Obtention of Liposomes Encapsulating Uric Acid Using HPLC Grade Water as Solvent

For the preparation of DSPC liposomes (18: 0 PC, or 1,2-distearoyl-sn-glycero-3-phosphocholine), PEG2000-PE (1,2-distearoyl-sn-glycero-3-phosphoethanolamine-n-[methoxy(polyethylene glycol)-2000) and cholesterol were used in the amounts indicated in the following table 3, for a total amount of lipids in the liposomes of 200 μmol.

TABLE 3
Molecular weight, molar fraction and total amount of
each of the liposome components to obtain 200 μmol
of liposomes in the method of the present invention.

| | Molecular weight | | |
|---|---|---|---|
| components | (g/mol) | Amount (mg) | Molar fraction |
| DSPC (18:0 PC) | 790.1 | 97.46 | 0.617 |
| 18:0 PEG2000 PE | 2805.5 | 28.06 | 0.050 |
| Cholesterol | 386.6 | 34.02 | 0.333 |

These components were provided by Avanti Polar Lipids with references 850365P, 880120P and 700001P.

### Phase 1: Lipid Film Formation

A pear-shaped reaction flask was cleaned with milliQ water (3×), followed by ethanol (3×) and acetone (3×). A rotary evaporator was cleaned by the same process as well as every other non-disposable glassware needed in the process. The glassware to be used was dried under nitrogen or argon gas stream. The amounts of lipids and cholesterol required following table 2 were weighed and transferred to the reaction flask, to which 7 mL of a mixture of CH₃Cl: MeOH (chloroform-methanol, 6: 1) were admixed until complete dissolution. The reaction flask was attached to a rotary evaporator in bath at 30° C. and 140 rpm for the complete evaporation of the organic solvent, wherein the pressure dropped down to 200 mbar (20 kPa). From this point on, the vacuum was increased very slowly to 10 mbar over a period of 10 minutes and then the flask with the lipid film was kept at maximum vacuum for 15 minutes. The vacuum was then slowly broken and the flask was placed for 1 hour under nitrogen gas stream to favor the total evaporation of organic solvents.

### Phase 2: Lipid Film Rehydration

A 10 mM solution of uric acid-lysine co-crystal was prepared solving 328.7 mg of solid crystals in 100 ml of HPLC grade water at room temperature.

The uric acid-lysine co-crystal starting material has a PXRD as shown FIG. 6.

The solution of the uric acid to be encapsulated was heated to 65° C., which is over the Tₘ of the DSPC (55° C.), maintaining this temperature throughout the process. The reaction flask with the lipid film was placed in a water bath until reaching 65° C. and then, 7 mL of the solution were added to the flask, stirring with 5-6 units of glass beads of 3 mm in diameter until the film completely dissolved. Stirring was maintained for 10 min, with the flask always in the bath to maintain the temperature at Tₘ.

### Phase 3: Liposome Extrusion

An extrusion system was heated to the same temperature (10 mL LIPEX Thermobarrel, Evonik), connected to a thermostatic water recirculation bath and a nitrogen gas stream, as the driving gas. With the extruder balanced at a temperature 65° C., the extruder was washed (3×) with excess of HPLC grade water. A 0.4-micron pore size polycarbonate membrane was mounted on the extruder (Millipore) and was wet with solvent (HPLC grade water). The extruder was filled with the aqueous lipid mixture, allowing the temperature to equilibrate during 10 min. A nitrogen gas stream was used to force extrusion of the mixture through the membrane. Sample was collected in a hot falcon tube, at a temperature of 65° C. The extrusion process was repeated on the 0.4-micron membrane once more. The process was repeated 4 times using a membrane of 0.2 microns pore size and, finally, the extrusion process was repeated 8 times using a 0.1-micron pore size membrane (in total, the extrusion process involves 2×0.4 microns+4×0.2 microns and 8×0.1 microns), to obtain nominal size liposomes of hydrodynamic diameter around 100 nanometers (Malvern DLS system).

### Phase 4: Liposome Filtration

A centrifugal filter unit (Amicon tube with 30 kDa cut-off membrane) was filled with HPLC grade water and filtered in a centrifuge (10 min at 3000 g and at room temperature). Filter washing with HPLC water was repeated for 2 additional times (3× wash in total). The extruded liposome solution was passed through the previously washed centrifugal filter unit and centrifuged (2 hours at 3000 g). The final volume of the liposome solution was adjusted to 5 ml. The obtained liposomes were characterized as shown in Table 4 and stored at 4° C. for a period of several months.

Table 4 shows the average values obtained from five different batches of liposomes, wherein the amount of uric acid in the liposomes was determined by ultraviolet-visible spectrophotometry (UV-VIS at 295 nm).

| | Hydrodynamic | | | Encapsulated |
|---|---|---|---|---|
| Sample | diameter (nm) | Polydispersity | pH | UA (mg/ml) |
| AU | 111.9 ± 13.6 | 0.159 ± 0.157 | 7.2 ± 0.3 | 1.49 ± 0.18 |
| liposomes | | | | |

### Example 3: Process of Obtention of Liposomes Encapsulating Uric Acid Using a Saline Solution as Solvent

In addition to the method of Example 2, the following four different aqueous solvents were tested for dissolving the uric acid co-crystals:
Phosphate Buffer Saline at 10 mM (PBS 1×)
Saline solution of 0.9% NaCl (B|BRAUN)
A 0.9% (w/v) solution of NaCl in HPLC grade water
A 0.9% (w/v) solution of KCl in HPLC grade water

The concentration of uric acid, pH of the obtained solution and solubility of uric acid in the described solvents resulted as described in Table 5. The data of the HPLC solution were obtained in Example 2.

TABLE 5
Concentration and mass of uric acid co-crystal, volume of HPLC grade water pH and solubility
of de uric acid in the solvent of saline aqueous solutions used in the liposome
manufacturing process of the state of the art.

| | Mass of co- | | Concentration | | |
|---|---|---|---|---|---|
| | crystal of uric | Volume | of UA co- | | |
| Solvent | acid (mg) | solvent (ml) | crystal (mM) | pH | Soluble |
| HPLC grade | 378.21 | 100 | 10 | 7.20 | Yes |
| water | | | | | |
| Phosphate Buffer | 378.21 | 100 | 10 | 7.60 | No |
| Saline 10 mM | | | | | |
| (PBS 1x) | | | | | |
| Saline Solution | 378.21 | 100 | 10 | 6.49 | No |
| B\|Braun (NaCl | | | | | |
| 0.9%) | | | | | |
| NaCl solution at | 378.21 | 100 | 10 | 7.54 | No |
| 0.9% (W/V) | | | | | |
| KCl solution at | 378.21 | 100 | 10 | 7.65 | No |
| 0.9% (W7V) | | | | | |

Except for HPLC grade water, uric acid co-crystal was partially insoluble at the concentration of 10 mM, resulting these solutions not valid for the preparation of liposomes as described in Example 2.

### Example 4. Characterization of the Liposomes Obtained by the Method of the Present Invention

Following the method of the present invention described in Example 1, 22 batches of uric acid liposomes (AU liposomes) and 5 batches of liposomes of identical composition, but with no uric acid inside (control liposomes) were prepared. Each of the preparations was characterized, as indicated below.

1) Size and z-Potential of Liposomes.

Liposomes size (in the form of hydrodynamic diameter) and Z potential determination was done by means of DLS, for which a Malvern z-sizer equipment was used, operated according to the manufacturer's instructions, at a temperature of 25° C. Briefly, for this, a 100-microliter sample was extracted from the final liposome solution, bringing it to 1 mL (dilution 1 to 10) in HPLC grade water that was introduced into a cuvette. The equipment was turned on and the laser was allowed to stabilize for at least 30 minutes. The sample was then thermostated in the cuvette inside the equipment for at least 3 minutes, to proceed later with the measurement. In no case the sample concentration was greater than 1 mg/mL.

The size (in the form of hydrodynamic diameter), polydispersity and potential Z obtained was indicated in Table 6 included below:

TABLE 6
Liposomes that encapsulate uric acid obtained according to
the method of the present invention and
control liposomes size and Z potential.

| | Hydrodynamic | | |
|---|---|---|---|
| Sample | diameter (in nm) | Polydispersity | Z Potential (mV) |
| AU liposomes | 111.9 ± 13.6 | 0.159 ± 0.157 | 12.17 ± 6.8 |

2) Production Performance

Is normal that in the multiple stages involved in the synthesis, extrusion and filtering of liposomes, a certain amount of material is lost. To determine the performance of the process of the present invention, the amount of lipids in the final liposome solutions was determined, using the well-known Rouser colorimetric method (Rouser G, Fkeischer S, Yamamoto A. Two dimensional then layer chromatographic separation of polar lipids and determination of phospholipids by phosphorus analysis of spots. Lipids. 1970 May; 5(5):494-6. doi: 10.1007/BF02531316. PMID: 5483450).

TABLE 7
Yield results in production obtained in Example 4.

| Sample | Heavy lipids (μmol) | Final lipids (μmol) | Yield (%) |
|---|---|---|---|
| AU liposomes | 133.35 ± 0.02 | 91.08 ± 8.3 | 68.3 ± 6.2 |

### 3) Encapsulation Efficiency

In each uric acid liposomes preparation, the exact amount of therapeutic agent encapsulated in the liposomes was calculated using a colorimetric technique based on the uricase digestion method described by Hamzah H H et al. (Hamzah H H, Zain Z M, Musa NLW, Lin Y C, Trimbee E (2013) Spectrophotometric Determination of Uric Acid in Urine Based-Enzymatic Method Uricase with 4-Aminodiphenylamine Diazonium Sulfate (Variamine Blue RT Salt). J Anal Bioanal Tech S7: 011. doi:10.4172/2155-9872.S7-011). In summary, 25 μL of non-encapsulated uric acid solution were taken (after encapsulation, the non-encapsulated uric acid sample is obtained in the filtration phase by centrifugation), 25 μL of Variamin 0.1 mM and 50 μL of uricase (50 μg/ml), bringing the final volume to 1 ml. The mixture was kept at 37° C. for 30 minutes and the amount of uric acid in solution was determined by measuring the absorbance at 261 nm, determining the concentration by means of a calibration line obtained from standard uric acid solutions measured in the same way.

To calculate encapsulation efficiency, the exact uric acid amount added in each liposome preparation was recorded in phase 2 of the liposome preparation process of the present invention (hydration of the lipid film) and the non-encapsulated uric acid amount was determined by colorimetry, from the obtained filtrate in phase 4 of the liposome preparation process of the present invention (filtrate in an Amicon tube with a cut-off point of 30 kDa). Obtained results are summarized in Table 8 included below:

TABLE 8
Yield results in encapsulation obtained in Example 4
for the liposome preparation process of the present invention.

| | Initial uric | Encapsulated uric | Encapsulation |
|---|---|---|---|
| Sample | acid (mg) | acid (mg) | Efficiency (%) |
| AU liposomes | 11.77 ± 0.15 | 10.16 ± 0.21 | 86.3 ± 3.6 |

### 4) Liposomes Stability

Finally, a study was carried out in which stability of the liposomes in solution at room temperature was observed for a period of 30 days. In this period, no turbidity or precipitate formation was observed. On the other hand, the liposomes were filtered every 3-7 days and the uric acid amount that remained encapsulated and which part had been released in solution were determined, using the colorimetric method described in the previous section. Obtained results showed that 7 days after the liposomes synthesis or elaboration, more than 90% of uric acid remained encapsulated while less than 10% had been released, the liposomes remaining, therefore, stable in solution. At 15 and 30 days from the liposomes synthesis or elaboration, the initial uric acid that remained encapsulated amounts were greater than 80%, demonstrating the great stability of the liposomes (see FIG. 1). It is important to note that despite the fact that at long storage times a small portion of uric acid is released in solution (released from liposomes), the solutions retained their transparency and uric acid crystallization was not observed.

### Example 5. Analysis of Different Compositions of the Lipid Bilayer in Liposomes that Encapsulate Uric Acid

Four different lipid compositions used in the process of the present invention were analyzed for the preparation of liposomes that encapsulate uric acid:
1—DOPE: cholesterol: 18: 0 PEG2000-PE Molar fractions (0.583: 0.333: 0.083)
2—DSPC: Cholesterol: 18: 0 PEG2000-PE Molar fractions (0.583: 0.3333: 0.083)
3—DOTAP: DSPC: Cholesterol: 18: 0 PEG2000-PE Molar fractions (0.3: 0.283: 0.33: 0.083)
4—DSPC: DC-Cholesterol: 18: 0 PEG2000-PE Molar fractions (0.617: 0.333: 0.050)
DOPE: 18:1 (Δ9-Cis) PC (DOPC) 1,2-dioleol-sn-glycero-3-phosphocholine
DOTAP: N-[1-(2,3-Dioleoyloxy) propyl]-N, N, N-trimethylammonium

The lipid bilayer compositions 1 to 3 showed non-optimal results of encapsulation and uric acid release and lower than those of 4, which did show optimal results of encapsulation and uric acid release. Specifically, it was observed that the kinetics of uric acid release from the liposomes to dissolution were faster in compositions 1 to 3, compared to 4, in such a way that 7 days after preparation less than 65% of the UA remained. encapsulated in the liposomes of formulations 1 to 3 (compared to 90% of formulation 4) and after 15 days less than 45% of the UA remained encapsulated in the liposomes of formulations 1 to 3 (compared to 80% of formulation 4).

### Example 6. Efficacy Analysis of Uric Acid Liposomes of the Present Invention in a Mouse Model of Cerebral Ischemia/Reperfusion

In this example, the liposomes of the present invention efficacy was analyzed in a mouse model of brain ischemia/reperfusion. The experimental details were as follows:
Species: Mouse C57BL/6 from the supplier “Laboratorios Janvier”; sex: males; age: 10 to 14 weeks.
Dose: 16 mg uric acid/kg mouse weight.
Ischemia/reperfusion: 30 min middle cerebral artery occlusion (MCAO) (monitored with laser Doppler) followed by 24 h of reperfusion.
Treatment: a) Intravenous infusion (20 minutes duration) of the different treatments. Treatment began 30 min after reperfusion. Treatments were administered blind.
Neurological test: At 24 h the neuroscore was performed.
Euthanasia: After neurological test, the animals were sacrificed. Blood samples were taken, and the brain was extracted which was cut out for staining with TTC (tetrazolium chloride) to measure the cerebral infarct volume.
Inclusion/exclusion criteria: All mice that had a drop in blood flow greater than 65% and a reperfusion greater than 70% received the treatment. Of these, animals were not included if the injection was not correct. Of the correctly administered animals, mice that did not develop an infarct or with a very small infarct (<10%), and mice that had an infarct outside the territory of the middle cerebral artery were excluded. Animals that died were counted but were not included in the study due to the lack of volume/neuroscore data at end point.
See summary of the experimental protocol included in FIG. 2.

The treatment was randomized, and the administration of the drugs was performed blind. The treatment groups were:
a) Liposomes that encapsulate uric acid diluted in saline solution (Lipo-UA) (Liposomes according to the present invention).
b) Empty liposomes diluted in saline solution (Lipo) (i.e., liposomes prepared according to the method of the present invention but with no uric acid).
c) Uric acid solution (dissolved in purified water (UA).
d) Corresponding Purified water as vehicle (V).

### Results:

Table 9 includes a summary of the animals included in each of the experimental groups:

TABLE 9
Summary of the mice included in each of the experimental
groups of Example 4. The numbers indicated in the
table refer in all cases to the number of mice.

| | Lipo-UA | Lipo | UA | V |
|---|---|---|---|---|
| Total | 18 | 20 | 16 | 18 |
| Excluded | 1 | 5 | 4 | 3 |
| Included | 16 | 15 | 12 | 13 |
| Mortality | 1 | 0 | 0 | 2 |
| Mortality % | 5.6% | 0% | 0% | 11% |

Analysis of the results was done in a blinded mode.

The obtained results for the cerebral infarction volume and neuroscore in the different experimental groups are summarized in FIGS. 3 and 4.

Administration of Lipo-UA produced a significant decrease (31.79%) in the volume of the cerebral infarct compared to the control group (Lipo) (see FIG. 3).

UA administration produced an also significant decrease (28.61%) in the cerebral infarct volume compared to the control group (vehicle; V) (see FIG. 3).

Treatment with Lipo-UA and treatment with UA caused a similar reduction in cerebral infarct volume, however, a tendency was observed for the effect to be greater with Lipo-UA (see FIG. 3).

Additionally, and surprisingly, treatment with Lipo-UA produced an improvement in neurological function as deduced from the significant reduction (25.6%) in the neuroscore test score. This effect was not observed in UA treatment (see FIG. 4)

Additionally, it was possible to corroborate that the liposomes of the present invention reached the blood capillaries of the brain effectively (see example 5 and FIG. 5).

Finally, the liposome manufacturing method effect on their effectiveness was also studied. For this, the same protocol indicated above was followed but with uric acid liposomes prepared according to the state of the art method and uric acid liposomes prepared according to the method of the present invention. The results obtained were those shown in Tables 7 and 8 included below:

TABLE 10
Results obtained with uric acid liposomes prepared
according to the state of the art method.

| | | Lipo- | Reduction | |
|---|---|---|---|---|
| | Lipo | UA | % | p |
| No. of mice | 7 | 7 | | |
| Cerebral infarct volume | 42.57 | 40.03 | 5.97 | p = 0.6983 |
| (mm³) | | | | |
| Cerebral infarction volume | 26.24 | 23.99 | 8.57 | p = 0.5590 |
| (% with respect to the | | | | |
| volume of the | | | | |
| corresponding hemisphere | | | | |
| (corrected for edema)) | | | | |
| Neuroscore | 11.29 | 9.71 | 13.96 | p = 0.4202 |

TABLE 11
Obtained results with uric acid liposomes prepared
according to the method of the present invention.

| | | Lipo- | Reduction | |
|---|---|---|---|---|
| | Lipo | UA | % | p |
| No. of mice | 10 | 9 | | |
| Cerebral infarct volume | 45.53 | 31.86 | 30.02 | p = 0.0215 |
| (mm³) | | | | |
| Cerebral infarction volume | 27.26 | 19.83 | 27.26 | p = 0.0222 |
| (% with respect to the | | | | |
| volume of the | | | | |
| corresponding hemisphere | | | | |
| (corrected for edema)) | | | | |
| Neuroscore | 9.60 | 7.89 | 17.82 | p = 0.1216 |

As shown in Tables 10 and 11, the method of the present invention makes it possible to obtain liposomes that encapsulate uric acid that show a greater therapeutic effect (in the form of a smaller volume of cerebral infarction and a better neuroscore) compared to uric acid liposomes obtained with the process of the state of the art.

### Example 7. Study of the Localization of Liposomes of the Present Invention in the Mouse Brain

In this case, the ischemic mice (obtained as indicated in Example 4) were administered liposomes of the present invention (with uric acid) or control liposomes (with saline solution), all of them with a green fluorescent protein (Dioc18), according to the liposome administration method set forth above. Two hours after reperfusion, euthanasia was carried out, the brain was fixed with 4% paraformaldehyde, and the tissue was processed for immunofluorescence and confocal microscopy. For this, coronal sections of the brain were made with vibratome (50 μm thick) that were cryoprotected in glycerol and stored at −20° C. Blood vessels were stained with the anti-Glut1 antibody followed by a secondary antibody AlexaFluor-556 (red). Nuclei are visualized with DAPI staining (blue). A confocal microscopy study (DragonFly) was carried out, making 1 μm planes to carry out a co-localization study with the ImageJ software (Colocalization threshold).

FIG. 5 shows a representative image that illustrates the presence of the green fluorescent protein Dioc18 in the capillary wall (red) after the liposomes that encapsulate uric acid and Dioc18 administration.

Therefore, Examples 1 to 5 demonstrate that the inventors of the present invention have been able to effectively obtain liposomes that encapsulate uric acid, that said liposomes are stable over time and are superior (superior therapeutic effect) for the treatment of stroke.

Additionally, the results collected in examples 1 to 5 demonstrate the surprising results obtained with the process of the present invention for the preparation of liposomes that encapsulate uric acid, both in terms of performance and stability, as well as a surprising superior therapeutic effect.

1. A liposome that encapsulates uric acid, wherein said liposome has a lipid bilayer comprising double-chain phospholipids and positively charged cholesterol, and wherein said liposome is unilamellar, wherein the uric acid is obtained by preparing a solution of co-crystals of uric acid as starting material without lithium or potassium ions.

2. The liposome according to claim 1, wherein the encapsulated amount of uric acid is between 6*10⁻²⁰ and 6*10⁻¹⁸ moles per liposome.

3. The liposome according to claim 1, wherein said liposome has a hydrodynamic diameter measured by Dynamic Light Scattering (DLS) in water at a temperature of 25° C. of between 80 and 140 nm.

4. The liposome according to claim 1, wherein said liposome has a positively charge surface and a Z potential measured by Dynamic Light Scattering (DLS) at a temperature of 25° C. greater than 0 and lower than 15 mV.

5. The liposome according to claim 1, wherein between 2.5% and 10% molar of the double-chain phospholipids are phospholipids with one or more polyethylene glycol chains that hinder the opsonization of blood liposomes attached to a polar head of said phospholipids; and between 90 and 97.5% molar of the double chain phospholipids are phospholipids with no polyethylene glycol attached to a polar head of said phospholipids.

6. The liposome of claim 5, wherein:
said double chain phospholipids with one or more polyethylene glycol chains attached to a polar head are selected from 16: 0 PEG2000 PE (or 1,2 dipalmitoyl-sn-glycero-3-phosphoethanolamine-N-[methoxy (polyethylene glycol)-2000]), 18: 0 PEG2000 PE (or 1,2-distearoyl-sn-glycero-3-phosphoethanolamine-N-[methoxy (polyethylene glycol)-2000]), 18: 0 PEG5000 PE (or 1,2-distearoyl-sn-glycero-3-phosphoethanolamine-N-[methoxy (polyethylene glycol)-5000]) or combinations thereof; and
said double chain phospholipids without polyethylene glycol attached to a polar head are selected from DSPC (18: 0 PC or 1,2-distearoyl-sn-glycero-3-phosphocholine), DPPC (16: 0 PC or 1,2-dipalmitoyl-sn-glycero-3-phosphocholine)), 17: 0 PC (or 1,2-diheptadecanoyl-sn-glycero-3-phosphocholine) or combinations thereof.

7. The liposome according to claim 1, wherein said positively charged cholesterol is dimethylaminoethane-cabamoyl-cholesterol (DC-cholesterol) hydrochloride.

8. The liposome according to claim 7, comprising a molar fraction ratio between double chain phospholipids and DC-cholesterol between 0.6: 0.4 and 0.75: 0.25.

9. A method for the manufacture of liposomes that encapsulate uric acid, comprising the steps of:
a. preparing a solution of co-crystals of uric acid with L-lysine in purified water;
b. adjusting the pH of the solution to a value between 7.2 and 7.4;
c) forming a lipid film;
d) rehydrating the lipid film formed in step c) with the uric acid solution of step b) to obtain liposomes;
e) extruding the liposomes of step d) using purified water as solvent; and
f) filtering the liposomes obtained in step e) using purified water as solvent, wherein steps c) to f) are carried out free of calcium ions.

10. A pharmaceutical composition comprising a liposome according to claim 1 and at least one pharmaceutically acceptable excipient.

11. A medicine comprising the pharmaceutical composition according to claim 10.

12. A method for improving and/or treating cerebrovascular disease comprising administering the pharmaceutical composition according to claim 10 to a patient in need thereof.

13. A medicine comprising the liposome according to claim 1.

14. A method for improving and/or treating cerebrovascular disease comprising administering the liposome according to claim 1 to a patient in need thereof.
